(19) United States
(12) Patent Application Publication
(10) Pub. No.: US 2007/0085995 A1
(43) Pub. Date: Apr. 19, 2007

(54) Wearable glucometer

(75) Inventors: Benny Pesach, Rosh-Ha'ayin (IL); Gabriel Bitton, Jerusalem (IL); Ron Nagar, Tel-Aviv (IL)

Correspondence Address: WOLF, BLOCK, SCHORR & SOLIS-COHEN LLP, 250 PARK AVENUE, NEW YORK, NY, 10177, US

(73) Assignee: Glucon, Inc., Boulder, CO (US)

(21) Appl. No.: 10/564,127

(22) PCT Filed: Jul. 9, 2004
(86) PCT No.: PCT/IL04/00619

§ 371 Date: Aug. 14, 2006

Related U.S. Application Data
(60) Provisional application No. 60/485,403, filed on Jul. 9, 2003.

(51) Int. Cl. G01N 33/48

(52) U.S. Cl. 356/39

(57) ABSTRACT

Apparatus for assaying an analyte in blood in a blood vessel below a patient's skin comprising: at least one light source controllable to transmit light into tissue below the skin through at least one first region on the skin; at least one light detector that receives a portion of the transmitted light that reaches at least one second region on the skin after propagating through the blood vessel and generates signals responsive to the received light; and a controller; wherein the controller controls the at least one light source to transmit light at least one wavelength that interacts with blood and at least one wavelength that interacts with the analyte and uses the signals responsive to the light that interacts with the blood to determine a location for the blood vessel and the determined location and signals responsive to the light to assay the analyte.

### RELATED APPLICATIONS

The present application claims the benefit under 35 USC 119(e) of US provisional application 60/485,403 filed on Jul. 9, 2003, the disclosure of which is incorporated herein by reference.

### FIELD OF THE INVENTION

The invention relates to monitoring apparatus, for example one that can be coupled to a body and continuously assay a substance in the body for an extended period of time and in particular wearable apparatus for continuously monitoring glucose levels in a body.

### BACKGROUND OF THE INVENTION

Methods and apparatus for determining blood glucose levels for use in the home, for example by a diabetic who must monitor blood glucose levels frequently, are available. These methods and associated devices are generally invasive and usually involve taking blood samples by finger pricking. Often a diabetic must determine blood glucose levels many times daily and finger pricking is perceived as inconvenient and unpleasant. To avoid finger pricking, diabetics tend to monitor their glucose levels less frequently than is advisable.

Non-invasive in-vivo methods and apparatus for monitoring blood glucose are known. PCT Publication WO 98/38904, the disclosure of which is incorporated herein by reference, describes a “non-invasive, in-vivo glucometer” that uses a photoacoustic effect to measure a person's blood glucose. PCT Publication WO 02/15776, the disclosure of which is incorporated herein by reference, describes locating a blood vessel in the body and determining glucose concentration in a bolus of blood in the blood vessel. In an embodiment described in the publication glucose concentration in the blood bolus is determined by illuminating the bolus with light that is absorbed and/or scattered by glucose to generate photoacoustic waves in the bolus. Intensity of the photoacoustic waves, which is a function of glucose concentration, is sensed and used to assay glucose in the bolus.

U.S. Pat. No. 6,630,673, the disclosure of which is incorporated herein by reference, describes a method for non-invasively determining concentration of an analyte, e.g. glucose, in a layer of tissue beneath the skin of a patient. The method involves introducing light into the tissue through a first location on the skin and measuring intensity of the light that travels through the tissue and reaches a second location on the skin for at least two different distances between the first and second locations. The intensities of light for the different distances are used to assay concentration of the analyte in the layer. The patent does not describe methods for limiting the assay to blood in a blood vessel.

Wearable devices for assaying glucose are known, are generally based on near-infrared (NIR) spectroscopic methods and usually comprise a light source and optical detector that are attached to the patient's finger, wrist or other part of the body. Wearable NIR devices for assaying glucose are described in U.S. Pat. No. 6,241,663 to Wu, et al. and U.S. Pat. No. 5,551,422, to Simonsen et al., the disclosures of which are incorporated herein by reference.

An apparatus for determining glucose levels is hereinafter referred to as a “glucometer”.

### SUMMARY OF THE INVENTION

An aspect of some embodiments of the present invention relates to a wearable glucometer that may be mounted to a patient's skin in alignment with a blood vessel in the patient's body and thereafter operates to repeatedly assay glucose in blood in the blood vessel without requiring substantial user intervention.

A glucometer in accordance with an embodiment of the present invention comprises at least one light source, at least one optical detector and a controller. When the glucometer is mounted to the skin of a patient, the controller controls the at least one light source to transmit light into tissue beneath the skin through at least one localized “input” region on the skin. The at least one detector generates signals responsive to intensity and/or phase of intensity modulation of light that reaches at least one localized “output” region on the skin for at least two different pairs of input and output regions for which distances between the input and output regions are different. These signals may be further processed and displayed as blood glucose values. The intensity and/or phase of intensity modulation of light that reaches an output region is generally a function of scattering, reflection, and/or absorption, of the transmitted light with subcutaneous tissue, and/or stimulation of light emission in the subcutaneous tissue, in a region between the output location and its corresponding input location. A region through which light propagates through subcutaneous tissue between an input region on the skin and an output region on the skin is hereinafter referred to as a “propagation channel”.

In accordance with an embodiment of the invention, the controller controls the at least one light source to transmit light through the skin between at least two pairs of input and output regions at a wavelength of light that is absorbed by blood. The controller uses signals generated by the at least one detector responsive to the intensities and/or phases of intensity modulation of the transmitted light that reaches the output regions to determine a location of a blood vessel beneath the skin relative to the position of the glucometer. Optionally, the light transmitted into the subcutaneous tissue is phase and/or amplitude modulated and the intensity and/or detector signals are processed responsive to the modulation of the transmitted light to locate the blood vessel Any of various methods known in the art for controlling the light sources and detectors to provide intensity and/or phase measurements suitable for determining a location of a feature beneath the skin responsive to the measurements may be used to determine location of the blood vessel Such methods are described for example in U.S. Pat. No. 6,272,3673, U.S. Pat. No. 6,630,673, U.S. Pat. No. 6,564,088 and in the “Handbook of Optical Biomedical Diagnostic Diagnostics”, Valery V. Tuchin, SPIE Press, 2002, SPIE Press, 2002, the disclosures of which are incorporated herein by reference.

In some embodiments of the invention, the flow of blood through the blood vessel is modulated and time dependence of intensity and/or phase of modulation signals provided by the at least one detector are correlated with time dependence of the blood flow modulation to locate the blood vessel. If a propagation channel defined by an input and output region on the skin passes through the blood vessel a portion of the light transmitted between the input and output regions will be modulated by the blood volume and/or velocity changes. The intensity and/or phase signals provided by the detector responsive to the light at the output region will reflect the modulation and indicate that the propagation channel associated with the input and output regions passes through the blood vessel In some embodiments of the invention, blood flow is modulated by applying periodic pressure changes to a region of the patient's body near to the location of the glucometer. In some embodiments of the invention, the periodic pressure changes are generated by ultrasound to which the blood vessel is exposed. A portion of light transmitted between an input and output region for which the associated propagation channel passes through the blood vessel is modulated by an acousto-optic effect generated by the ultrasound.

Optionally, the controller uses the location of the blood vessel to aid in aligning the field of view of the glucometer with the blood vessel. The locations of input and output regions on the skin for a given location of the glucometer are determined by a spatial configuration of the at least -one light source and at least one detector and/or optical components that optionally transport light respectively from and to the light source and detector. For glucometers for which the light source and detector, and/or associated optical elements are moveable, the locations of input and output regions may also depend on ranges over which the light source, detector and associated optical elements are moveable. The volume of subcutaneous tissue defined by the propagation channels between the input and output regions of the glucometer is defined as the field of view of the glucometer.

Optionally, the controller generates a signal responsive to the location of the blood vessel to aid a user of the glucometer to align the glucometer field of view with the blood vessel. Optionally, the glucometer comprises a display screen and the controller and processes intensity and/or phase signals generated by the at least one detector to generate and display an image of the blood vessel, an icon and/or another indication responsive to the detection to facilitate aligning the glucometer with the blood vessel.

In some embodiments of the invention, the glucometer is self-aligning and the controller adjusts position of the at least one light source and/or at least one detector responsive to the location of the blood vessel to align the glucometer with the blood vessel. Self-aligning glucometers are described in PCT Application PCT/IL2004/000483, the disclosure of which is incorporated herein by reference, and a self-aligning glucometer in accordance with the present invention may comprise any of the devices and employ any of the methods described in the application to align itself with a blood vessel.

Once aligned, the controller controls the at least one light source to transmit light at at least one wavelength, hereinafter a “mensuration wavelength”, for which light is absorbed and/or scattered by glucose between at least one pair of input and output regions on the patient's skin for which the associated propagation channel passes through the blood vessel. Optionally, the controller controls the at least one light source and at least one detector to measure the intensity and/or phase of intensity modulation of mensuration light that propagates between at least one pair of input and output regions for which the propagation channel substantially does not pass through the blood vessel. A propagation channel between an input and output region that passes through the blood vessel is referred to as an “assay propagation channel” and a propagation channel that substantially does not pass through the blood vessel is referred to as a “reference propagation channel”.

Intensity and/or phase signals generated by the at least one detector for mensuration light that propagates through the assay propagation channel are used to determine an attenuation length for the mensuration light in tissue of the assay region. Intensity and/or phase signals generated by the at least one detector for mensuration light that propagates through the reference propagation channel are used to determine an attenuation length for the mensuration light for tissue in the reference region. Any of methods known in the art, such as those described in U.S. Pat. No. 6,272,3673, U.S. Pat. No. 6,630,673, U.S. Pat. No. 6,564,088 and the “Handbook of Optical Biomedical Diagnostic Diagnostics”, referenced above may be used to determine the attenuation lengths from the intensity and/or phase signals provided by the at least one detector.

The attenuation lengths for tissue in the assay and reference regions are optionally processed using methods known in the art to determine an attenuation length for blood in the blood vessel and therefrom an assay of glucose in the blood. Optionally, methods of assaying an analyte responsive to attenuation lengths described in PCT Application PCT/IL2004/000289, the disclosure of which is incorporated herein by reference, are used to assay glucose in blood in the blood vessel.

There is therefore provided in accordance with an embodiment of the invention, apparatus for assaying an analyte in blood in a blood vessel below a patient's skin comprising: at least one light source controllable to transmit light into tissue below the skin through at least one first region on the skin; at least one light detector that receives a portion of the transmitted light that reaches at least one second region on the skin after propagating through the blood vessel and generates signals responsive to the received light; and a controller; wherein the controller controls the at least one light source to transmit light at at least one wavelength that interacts with blood and at at least one wavelength that interacts with the analyte and uses the signals responsive to the light that interacts with the blood to determine a location for the blood vessel and the determined location and signals responsive to the light to assay the analyte.

Optionally, the controller controls the at least one light source and/or the at least one detector to transmit light between at least two pairs of first and second regions on the skin for which the distance between the first and second regions in one pair is different from that of the other pair.

Additionally or alternatively, the apparatus comprises modulating apparatus that modulates the flow of blood through the blood vessel and thereby causes corresponding modulation of the signals. Optionally, the modulation apparatus comprises an ultrasound transmitter that illuminates the blood vessel with ultrasound. Additionally or alternatively, the modulation apparatus comprises a source of electrical power that applies a time varying electric field to a region of the patient's body that causes recurrent tensing and relaxation of muscles that affect the size of the blood vessel. In some embodiments of the invention, the modulation apparatus comprises a mechanical resonator that applies a time varying pressure to a region of the blood vessel.

In some embodiments of the invention, the controller determines the location responsive to the modulation of the signals. In some embodiments of the invention, the controller assays the analyte responsive to the modulation of the signals.

In some embodiments of the invention, the apparatus comprises a light pipe coupled to each of the at least one light source that transmit light from the light source to the at least one first region. In some embodiments of the invention, the apparatus comprises a light pipe coupled to each of the at least one light detector that transmit light from the at least one second region to the detector.

In some embodiments of the invention, the at least one detector comprises a plurality of detectors. Optionally, the detectors comprise pixels in a CCD. Optionally, the apparatus comprises a lens that collects light from the at least one second region and focuses the light on the CCD.

In some embodiments of the invention, the light source comprises a single light source. In some embodiments of the invention, the light source is controllable to be moved so as to illuminate different first regions on the skin. In some embodiments of the invention, the analyte is glucose.

There is further provided in accordance with an embodiment of the invention, method for assaying an analyte in blood in a blood vessel below a patient's skin comprising: transmitting light at at least one wavelength that interacts with blood and at at least one wavelength that interacts with the analyte into tissue below the skin through at least one first region on the skin; generating signals responsive to a portion of the transmitted light at each of the at least one wavelengths that reaches at least one second region on the skin after propagating through the blood vessel; using the signals responsive to the light that interacts with the blood to determine a location for the blood vessel and the determined location and signals responsive to the light to assay the analyte.

### BRIEF DESCRIPTION OF FIGURES

Non-limiting examples of embodiments of the present invention are described below with reference to figures attached hereto, which are listed following this paragraph. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1A schematically shows a perspective view of a glucometer assaying glucose in a blood vessel in accordance with an exemplary embodiment of the present invention;

FIG. 1B schematically shows a cross-section view of a portion of the glucometer shown in FIG. 1A, in accordance with an exemplary embodiment of the present invention;

FIG. 2A schematically shows another glucometer assaying glucose in a blood vessel in accordance with an exemplary embodiment of the invention; and

FIGS. 2B and 2C schematically show side views of the glucometer shown in FIG. 2A.

### DETAILED DESCRIPTION OF EXEMPLARY EMBODIMENTS

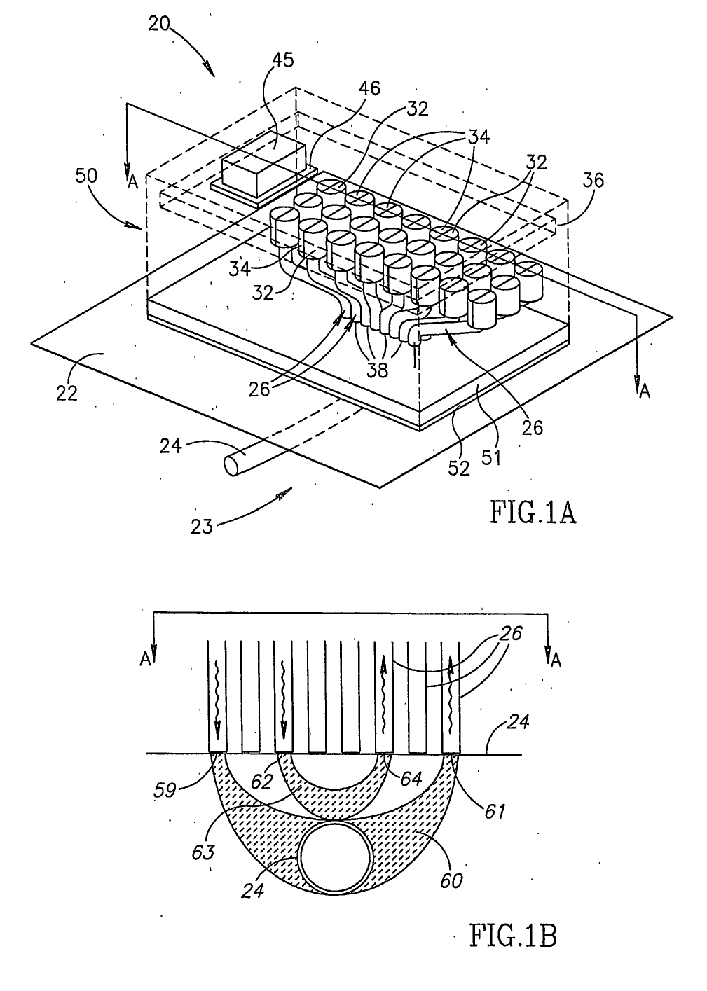

FIG. 1A schematically shows a perspective view of a glucometer 20 in accordance with an embodiment of the present invention. Glucometer 20 is shown attached to a region of skin 22 of a patient after the glucometer has been aligned with a blood vessel 24 located in a tissue region 23 under the patient's skin in order to assay glucose in blood in the blood vessel. FIG. 1B shows a cross section view of a portion of glucometer 20 and blood vessel 24 taken in a plane indicated by a line “AA” in FIG. 1A. Aligning glucometer 20 with blood vessel 24 is discussed below.

Components of glucometer 20 are comprised in a housing 50, shown in dashed lines, which comprises a mounting plate 51 that is adhered to skin 22 optionally using an adhesive 52 to secure the glucometer to skin 22 Optionally glucometer 20 is attached to a region of the wrist. Optionally the glucometer is attached using a strap. Optionally, a power source 45 for powering components of glucometer 20 and a controller 46 is mounted inside the housing. In some embodiments of the invention glucometer 20 receives power from an external power source optionally mounted to the patient's body. Housing 50 optionally comprises a visual display screen (not shown) and control buttons (not shown) for feedback and/or for transmitting commands and or data to controller 46.

Glucometer 20 optionally comprises a plurality of light pipes 26 each of which is coupled to a light source 32 and a light detector 34 using any of many methods available in the art and has an end 38 coupled to mounting plate 51 so that when the mounting plate is adhered to skin 22 end 38 is in optical contact with the skin. Optionally, sources 32 and detectors 34 are formed in a suitable substrate 36 using micro-manufacturing techniques known in the art. Whereas glucometer 20 comprises 24 light pipes 26 and ends 38 of the light pipes are configured in a rectangular array comprising three rows and eight columns, glucometer 20 may comprise a different number of light pipes and ends of the light pipes may be configured in arrays other than rectangular.

Controller 46 controls light sources 32 to selectively transmit or not transmit light, and receives signals generated by detectors 34 responsive to light incident on the detectors. When the controller controls light source 32 coupled to a given light pipe 26 to transmit light, the light illuminates tissue below skin 22 through a localized region on the skin directly below end 38 of the light pipe. Optionally, end 38 is formed with a lens (not shown) and/or coupled to optics (not shown) formed in mounting plate 51 that shapes light from light source 32 that exits end 38 into a beam of light (not shown) having a desired shape. Optionally, the lens and/or optics shapes the light into a collimated pencil beam. Signals that the controller receives from detector 34 which is coupled to the given light pipe 26 are generated responsive to light transmitted into tissue 23 by another light pipe 26. The light transmitted by the other light pipe reaches end 38 of the given light pipe through the region of skin 22 directly below the end of the given light pipe. When light is transmitted by light pipe 26, the region directly below the light pipe's end 38 functions as an optical input region on the skin. When light is received by the light pipe through the region opposite its end 38, the region functions as an optical output region on the skin.

Light that is transmitted into tissue 23 through an optical input region on skin 22 by a first light pipe 26 and propagates through the tissue to an optical output region on skin 22 where it is received by a second light pipe 26, propagates within a relatively well-defined “banana shaped” region in the tissue. FIG. 1B schematically shows a cross section of banana shaped propagation channels 60 and 63 for two pairs of light pipes 26. By way of example, propagation channel 60 extends from an input region 59 to an output region 61 on skin 22 and intersects blood vessel 24 and propagation channel 63 extends from an input region 62 to an output region 64 on the skin and does not intersect the blood vessel. Because propagation channel 60 intersects blood vessel 24 light that propagates between input region 59 and output region 61 interacts with blood in the blood vessel and is affected by optical properties of the blood and the propagation channel is an assay propagation channel. Because propagation channel 63 does not intersect blood vessel 24, light that propagates between input and output regions 62 and 64 and is not affected by blood in blood vessel 24, is affected by optical properties substantially only of tissue surrounding the blood vessel and is a reference propagation channel

It is noted that whereas assay propagation channel 60 is centered on blood vessel 24, the definition of an assay propagation channel is not a strict definition and a propagation channel is accepted as an assay propagation channel if blood in blood vessel 24 contributes substantially to the attenuation length for glucose in tissue in the propagation channel

It is noted that the banana shape configurations of the propagation channels for light transmitted through subcutaneous tissue between input and output regions on the skin are well known and may, for example, be modeled under varying assumptions of the optical properties of the tissue using Monte Carlo simulations and/or diffusion calculations. In general as the distance between an optical input region and an optical output region increase, the banana shape propagation channel dips deeper below the skin and light transmitted between the input and output regions samples tissue at greater depths below the skin.

To assay glucose in blood in blood vessel 24, controller 46 controls at least one first light source 32 to transmit light at at least one mensuration wavelength into tissue region 23 and receives signals from at least one detector 34 responsive to transmitted mensuration light that has traversed an assay propagation channel such as region 60 shown in FIG. 1B, in the tissue. Optionally controller 46 receives signals from at least one detector 34 responsive to mensuration light that has traversed at least one reference propagation channel, such as propagation channel 62, in tissue 23. Controller 46 processes the signals to determine attenuation lengths for tissue in the assay and reference propagation channels. In general, assay propagation channels, e.g. assay propagation channel 60, comprise tissue, “background tissue”, surrounding blood vessel 24 as well as blood in the blood vessel. As a result, the attenuation length determined for the assay region is not equal to the attenuation length for blood only but is a function of an attenuation length for background tissue as well.

In accordance with an embodiment of the invention, the attenuation length determined for the at least one reference propagation channel and known or calculated geometries of the propagation channels are used to remove the contribution to the attenuation length determined for the at least one assay propagation channel and determine an attenuation length for blood in blood vessel 24. The attenuation length thus defined for blood in blood vessel 24 for at least one mensuration wavelength of light is used to assay glucose in the blood.

It is noted that controller 46 “knows” from the spatial configuration of glucometer 20 and the location of blood vessel 24 (which is aligned with the glucometer), which propagation channels in tissue region 23 defined by ends 38 of light pipes 26 are assay propagation channels and which are reference propagation channels. In some embodiments of the invention, a volume of blood flowing through blood vessel 24 is modulated and the modulation aids controller 46 in identifying which propagation channels are assay and which are reference propagation channels. For example, assuming that blood flow through blood vessel 24 is harmonically modulated, signals generated by detectors 34 responsive to light that propagates through a propagation channel that intersects blood vessel 24 will exhibit a corresponding harmonic modulation Furthermore, to an extent that blood vessel 24 intersects a greater region of the propagation channel, modulation of the light is stronger. Propagation channels for which blood vessel 24 marginally overlaps blood vessel 24 will exhibit relatively weak modulation.

In some embodiments of the invention periodic pressure is applied to the patient's body in a neighborhood of blood vessel 24 to modulate blood flow in blood vessel 24. Optionally, the pressure is applied using a mechanical resonator or an acoustic actuator such as is used in speakers. In some embodiments of the invention, an electric field is applied to tissue in the region of glucometer 20 to cause recurrent constriction and relaxation of muscles that affect the size of blood vessel 24.

In some embodiments of the invention, blood vessel 24 is illuminated with ultrasound to generate an acousto-optic effect that modulates light that passes through blood vessel 24. For example, by focusing or directing ultrasound waves into blood vessel 24 and synchronizing the measurement of the light output modulation with it, the modulation of a region in the blood can be isolated by choosing the delay between the ultrasound transmission and a light modulation time slot n accordance with the depth of the blood vessel and the tissue sound velocity.

As noted above, glucometer 20 is aligned with blood vessel 24 when the blood vessel, as shown in FIGS. 1A and 1B, is located substantially at the center of the field of view of glucometer 20The field of view of a glucometer 20 is a volume in tissue 23 that includes substantially all propagation channels defmed by ends 38 of light pipes 26. In addition, for glucometers for which the field of view has a long direction, such as the field of view of glucometer 20 which is defined by the rectangular array of light pipes 26, a blood vessel is optionally aligned with the glucometer so that the length of the blood vessel is substantially perpendicular to the long direction of the field of view. Aligning the blood vessel perpendicular to the long direction can reduce sensitivity of assays provided by the glucometer to displacements of the glucometer perpendicular to the blood vessel.

In an exemplary embodiment of the invention, to align glucometer 20 with blood vessel 24, glucometer 20 is placed on a region of skin 22 below which blood vessel 24 is expected to be located. A suitable gel or oil is optionally used to optically couple the glucometer to the skin. A control signal is input to glucometer 20, for example via interface buttons comprised in the glucometer, instructing controller 46 to operate in an alignment mode and the glucometer is oriented so the long dimension of the rectangular array of light pipe ends 38 is perpendicular to blood vessel 24.

The patient, and/or a person aiding the patient, then moves glucometer 20 back and forth substantially in a direction perpendicular to the length of blood vessel 24. Optionally, during motion of glucometer 20, controller 46 controls light sources 32 to transmit light into tissue 23 that is, optionally, strongly absorbed and/or scattered by blood in blood vessel 24 and receives signals responsive to the transmitted light that propagate through different propagation channels defined by ends 38 of light pipes 26. Controller 46 processes the signals using methods known in the art to determine a location for blood vessel 24 and/or to generate an “alignment” image of features in tissue 23.

Optionally, controller 46 generates a signal responsive to the location of blood vessel 24 and/or the image to aid a user of glucometer 20 to align the glucometer with the blood vessel. For example, controller 46 may control a LED and/or a small speaker (not shown) responsive to the location of blood vessel 24 relative to glucometer 20 to provide a visual and/or audio signal indicating when glucometer 20 is aligned with blood vessel 24.

Optionally, controller 46 displays the alignment image on a display screen optionally comprised in glucometer 20 to facilitate aligning the glucometer with the blood vessel For example, in some embodiments of the invention, controller 46 displays the image on the display screen together with a suitable fiducial mark representing the center of the field of view of glucometer 20. The patient, and/or the patient's aid, aligns glucometer 20 with blood vessel 24 responsive to a location in the image of blood vessel 24 relative to the fiducial mark.

Once the glucometer is substantially aligned with blood vessel 24, the position of the aligned glucometer on the patient's skin is optionally marked using any suitable marking device, such as a pen for marking skin with non-toxic ink. The patient then removes glucometer 20 from skin 22 and optionally applies a layer of adhesive 26 to mounting plate 32 or removes a protective coating on a layer of adhesive 26 already in place on the mounting plate. The patient, and/or the patient's aid, then repositions glucometer 20 on skin 22 responsive to the alignment marks with the adhesive in contact with the skin and presses the glucometer to the skin to assure proper contact of the skin to the adhesive. Various methods of aligning a glucometer with a blood vessel are described in PCT Application PCT/IL2004/000483, the disclosure of which is incorporated herein by reference, and may be used in the practice of the invention

In some embodiments of the invention, glucometer similar to glucometer 20 is attached to a region of a patient's bloody without use of an adhesive and is held in place in close contact or contiguous with the skin by a strap. In some embodiments of the invention a glucometer similar to glucometer 20 is not worn, but is periodically, as required, held in place on a region of a patient' body to provide a glucose measurement.

It is noted that once aligned, glucometer 20 optionally repeatedly checks the location of blood vessel 24 by illuminating tissue 23 with light that interacts, optionally strongly, with blood and processing signals generated by detectors 34 to determine an “updated” location of the blood vessel. Controller 46, processes signals generated by detectors 34 responsive to light that light sources 32 provide at a wavelength that interacts with glucose in accordance with the updated location of blood vessel 24.

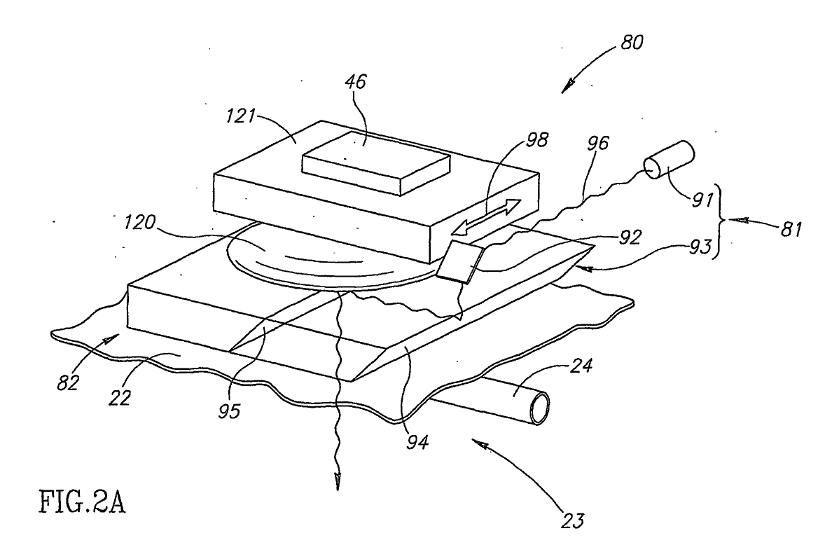

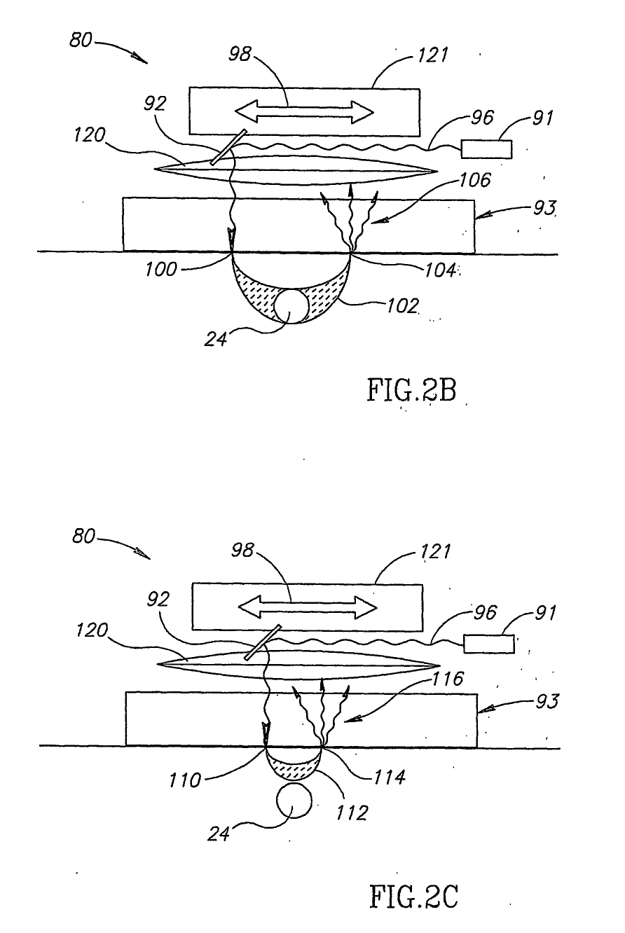

FIG. 2A schematically shows a perspective view of a glucometer 80 assaying blood in blood vessel 24 in a tissue region 23 below skin 22, in accordance with an alternative exemplary embodiment of the present invention. FIGS. 2B and 2C show side views of glucometer 80. Only features of glucometer 80 germane to the discussion are shown in FIG. 2A-2C

Glucometer 80 comprises an illumination system 81 for inserting light into tissue 23 at different optical input regions on skin 22 and an imaging system 82 for imaging light that propagates through tissue 23 to different optical output regions on skin 22 and a controller 46. Illumination system 81 optionally comprises a light source 91, a directing mirror 92 and a prism 93 having a strip reflecting strip facet 94 and a partially reflecting surface 95 parallel to strip facet 94. Light source 91 provides light 96 which is incident on directing mirror 92. The directing mirror reflects the incident light to facet 94, which reflects the light it receives to partially reflecting surface 95. Reflecting surface 95 directs a portion of the light it receives to skin 22 and into tissue 23 below the skin. Directing mirror 92 is controllable by controller 46 to be moved back and forth along directions indicated by double headed block arrow 98 and be positioned at different locations along a line parallel to strip facet 94. As a result, by positioning directing mirror 92 at different positions along strip facet 94 light 96 can be inserted into tissue 23 through skin 22 at different optical input regions on the skin lying along a straight line parallel to partially reflecting surface 95. In FIG. 2A light 96 is shown incident on skin 22 at an optical input region 100 on the skin. FIG. 2B shows a side view of glucometer .80 in which light 96 is incident on optical input region 100 shown in FIG. 2A In FIG. 2B directing mirror 92 ahs been moved closer to light source 91 by controller 46 and light 96 is incident on an optical input region 110 on skin 22.

Imaging system 82 comprises optionally a CCD 121 and optionally an optical transfer system, represented by a lens 120, that conveys light to the CCD. In some embodiments of the invention, optical system 120 comprises a plurality of lenses each of which images a different region of skin 22 on CCD 121. In some embodiments of the invention, optical system 120 10 comprises at least one collimating element that collimates light from regions of the skin propagating towards CCD121. A portion of light 96 that propagates from an optical input region on skin 22, such as optical input regions 100 and 110, to an optical output region on skin 22 is transmitted through partially reflecting surface 95 and prism 93 towards lens 120. Lens 120 collects the light from the output regions and focuses the light on CCD 121. Each pixel (not shown) in CCD 121 defines and images a different optical output region of skin 22 and generates signals responsive to the light that it receives. By way of example, FIG. 2B schematically shows an. assay propagation channel 102 that extends from input region 100 to an output region 104 and light 106 propagating from the output region towards lens 120 and CCD 121. FIG. 2C schematically shows a reference propagation channel 112 that extends from input region 110 to an output region 114 and light 116 propagating from the output region towards CCD 121. For each position of directing mirror 92 and therefore, for each optical input region on skin 22, glucometer 20 simultaneously provides data for a plurality of propagation channels through tissue 23 equal to a number of pixels in CCD 121. Controller 46 receives the signals and processes them similarly to way in which controller 46 in glucometer 20 processes signals provided by detectors 34 to align glucometer 20 with blood vessel 24 and assay glucose in blood in the blood vessel.

Whereas, in glucometer 80 imaging system 82 comprises a lens 120, a glucometer similar to glucometer 80 in accordance with some embodiments of the invention, does not include a lens 120 and CCD 121 is positioned close to, or in contact with, prism 93 and light from skin 22 is imaged directly on the CCD without a lens.

In some embodiments of the invention, a glucometer similar to a glucometer described herein is used not only to monitor a patient's blood glucose but also to control the patient's blood glucose. The glucometer is connected to a suitable insulin delivery system, such as for example an insulin pump coupled to a needle or a drug delivery patch, controllable to administer insulin to a patient. The glucometer and delivery system are mounted to the patient's body. The glucometer controller controls the delivery system to administer insulin to the patient and control thereby the patient's blood glucose level responsive to blood glucose measurements provided by the glucometer.

It is noted that whereas the glucometers discussed above are described as being used to assay glucose, the glucometers may be used to assay an analyte in blood in a blood vessel other than glucose, such as triglycerides oxygenated hemoglobin, hematocrit. To assay an analyte in a blood vessel other than glucose, a glucometer in accordance with the invention is operated similarly to the way in which it is operated to assay glucose but with the glucometer's light source or sources providing light that is absorbed and/or scattered by the other analyte.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. Apparatus for assaying an analyte in blood in a blood vessel below a patient's skin comprising:
at least one light source controllable to transmit light into tissue below the skin through at least one first region on the skin;
modulating apparatus that modulates the flow of blood through the blood vessel;
at least one light detector that receives a portion of the transmitted light that reaches at least one second region on the skin after propagating through the blood vessel and generates signals responsive to the received light and the modulation; and
a controller;
wherein the controller controls the at least one light source to transmit light at at least one wavelength that interacts with blood and at at least one wavelength that interacts with the analyte and uses the signals responsive to the light that interacts with the blood to determine a location for the blood vessel and the determined location and signals responsive to the light to assay the analyte.

2. Apparatus according to claim 1 wherein the controller controls the at least one light source and/or the at least one detector to transmit light between at least two pairs of first and second regions on the skin for which the distance between the first and second regions in one pair is different from that of the other pair.

3. (canceled)

4. Apparatus according to claim 1 wherein the modulation apparatus comprises an ultrasound transmitter that illuminates the blood vessel with ultrasound.

5. Apparatus according to claim 1 wherein the modulation apparatus comprises a source of electrical power that applies a time varying electric field to a region of the patient's body that causes recurrent tensing and relaxation of muscles that affect the size of the blood vessel.

6. Apparatus according to claim 1 wherein the modulation apparatus comprises a mechanical resonator that applies a time varying pressure to a region of the blood vessel.

7. Apparatus according to claim 1 wherein the controller determines the location responsive to the modulation of the signals.

8. Apparatus according to claim 1 wherein the controller assays the analyte responsive to the modulation of the signals.

9. Apparatus according to claim 1 and comprising a light pipe coupled to each of the at least one light source that transmit light from the light source to the at least one first region.

10. Apparatus according to claim 1 and comprising a light pipe coupled to each of the at least one light detector that transmit light from the at least one second region to the detector.

11. Apparatus according to claim 1 wherein the at least one detector comprises a plurality of detectors.

12. Apparatus according to claim 11 wherein the detectors comprise pixels in a CCD

13. Apparatus according to claim 12 and comprising a lens that collects light from the at least one second region and focuses the light on the CCD.

14. Apparatus according to claim 1 wherein the light source comprises a single light source.

15. Apparatus according to claim 1 wherein the light source is controllable to be moved so as to illuminate different first regions on the skin.

16. Apparatus according to claim 1 wherein the analyte is glucose.

17. A method for assaying an analyte in blood in a blood vessel below a patient's skin comprising:
modulating the flow of blood through the blood vessel;
transmitting light at at least one wavelength that interacts with blood and is modulated responsive to the modulation and at at least one wavelength that interacts with the analyte into tissue below the skin through at least one first region on the skin;
generating signals responsive to a portion of the transmitted light at each of the at least one wavelengths that reaches at least one second region on the skin after propagating through the blood vessel;
using the signals responsive to the light that interacts with the blood to determine a location for the blood vessel and the determined location and signals responsive to the light to assay the analyte.
